(19) United States
(12) Patent Application Publication
(10) Pub. No.: US 2024/0168014 A1
(43) Pub. Date: May 23, 2024

(54) METHOD FOR THE IN VITRO DIAGNOSIS OF INFECTION

(71) Applicant: ORIGINAL G B.V., Groningen (NL)

(72) Inventors: Joost Alexander Christiaan GAZENDAM, Groningen (NL); Matthew Francis BURTON, Groningen (NL)

(21) Appl. No.: 18/550,626

(22) PCT Filed: Mar. 21, 2022
(86) PCT No.: PCT/NL2022/050149

§ 371 Date: Sep. 14, 2023

(30) Foreign Application Priority Data
Mar. 19, 2021 (NL) 2027785

(51) Int. Cl. G01N 33/569; G01N 33/542; G01N 33/543

(52) U.S. Cl. CPC G01N 33/56911; G01N 33/542; G01N 33/54313

(57) ABSTRACT

The present invention relates to a method for in vitro diagnosing infection in bodily fluid samples, a lyophilized bead comprising a reagent and carbohydrate, the use of a lyophilized bead comprising a reagent and carbohydrate in an enzymatic assay, a system for detecting the presence of infection in a bodily fluid and a kit for detecting the presence of infection in a bodily fluid.

### FIELD OF THE INVENTION

The present invention relates to a method for in vitro diagnosing infection in bodily fluid samples, a lyophilized bead comprising a reagent and carbohydrate, the use of a lyophilized bead comprising a reagent and carbohydrate in an enzymatic assay, a system for detecting the presence of infection in a bodily fluid and a kit for detecting the presence of infection in a bodily fluid.

### BACKGROUND ART

There is an increasing clinical need for improved diagnostic methods for detecting infection in bodily fluid samples. With the rise of antibiotic resistance, it is becoming increasingly important to diagnose infection at an early stage before biofilms develop. A biofilm is a layer comprising any syntrophic consortium of microorganisms in which cells stick to each other and often also to a surface of a medical device. Such a layer is difficult to treat with antibiotics. Enabling early diagnosis of an infection can slow biofilm formation/improve the efficacy of antibiotic treatment.

A particular need for rapid infection diagnosis is in the area of implanted medical devices. For example, with infections associated with joint replacement. Approximately 1.5 million total hip replacement (total hip arthroplasty—THA) operations are carried out world-wide annually. This is likely to increase to approximately 3 million worldwide per annum within the next decade. In addition, other types of implants and joint replacements, such as knee, shoulder, foot, ankle, hand, wrist, elbow, cranio-maxillofacial and dental, are also being used in increasing amounts. Globally, the infection rate for THA is 1-16%, typically in the USA and Europe the infection rate is 3-12%, although in more elderly patient groups the infection rate for THA rises to 24%. Early, post-operative diagnosis of infection of implanted medical devices is desired in order to avoid difficult to treat infections and revision surgery.

In addition, known tests e.g. Synovaure® (Zimmer Biomet) aimed at identifying the presence of an infection such as periprosthetic joint infection measure biomarkers of the host (human) immune system response to infection, and thus do not directly detect infections (bacteria). A downside of indirect testing directed to host (human) immune system response is that in the acute phase (first couple of weeks post operation) it is not possible to differentiate from host (human) immune system inflammatory response and the host (human) immune system infection response.

Likewise, medical devices that are implanted in the spine are also prone to infection. Postoperative spine infection can be a devastating complication after spine surgery in both the short term and long term. Infection places a patient at a high risk for pseudoarthrosis, chronic pain, return to operating room, adverse neurological sequelae, worsened long-term outcomes, and even death. Depending on the type of spine surgery being performed, the incidence of infection is highly variable, with reported ranges listed up to 18%. Posterior cervical surgery has a higher rate of infection than posterior lumbar surgery and anterior spinal surgery.

Another clinical group in need of rapid infection diagnosis are patients undergoing peritoneal dialysis. Peritoneal dialysis (PD) has advantageous over haemodialysis, for example provision of peritoneal dialysis is generally less expensive and has fewer negative side effects (such as nausea, vomiting, cramping, and weight gain). However, the disadvantage of PD is peritonitis. Peritonitis is a common complication of peritoneal dialysis. It's associated with significant morbidity, catheter loss, transfer to haemodialysis, transient loss of ultrafiltration, possible permanent membrane damage, and occasionally death. Peritonitis may be directly related to peritoneal dialysis or secondary to a non-dialysis-related intra-abdominal or systemic process. Most cases are peritoneal dialysis related. Peritoneal dialysis-related peritonitis is either due to contamination with pathogenic skin bacteria during exchanges (i.e., touch contamination), or to an exit-site or tunnel infection. Secondary peritonitis is caused by underlying pathology of the gastrointestinal tract and occasionally (albeit rarely) due to haematogenous spread (i.e., following dental procedures). Causes of secondary peritonitis include cholecystitis, appendicitis, ruptured diverticulum, treatment of severe constipation, bowel perforation, bowel ischemia, and incarcerated hernia. Rapid diagnosis of peritonitis is important in order begin antibiotic treatment as early as possible.

Currently, bodily fluid samples need to be subject to complex, time consuming analytical techniques or microbiological assays, which means that a rapid diagnosis cannot be carried out at the point of care. Use of lateral flow devices is hampered by sample inhomogeneity and contamination with blood in traumatic samples.\

WO 2016/076707 discloses an object surface coating in which the cleavage of the first cleavage site simultaneously results in the release of the first non-quenching agent from the object surface coating so that emission of the first fluorescent agent can be detected. WO 2016/076707 teaches a heterogenous system for detecting infection in vivo. WO 2016/076707 does not relate to an in vitro diagnostic method.\

WO 2018-224561 teaches a method for detecting food spoilage microbes the method comprising:
a) adding a first pH adjustment agent to a food sample to provide a food sample having a pH in the range of pH 1 to 5, separating any solid precipitate present in the pH adjusted food sample to provide a pH adjusted food sample and adding a second pH adjustment agent to the pH adjusted food sample to provide a food sample having a pH in the range of pH 6.5-9 to be used in step b),\
b) contacting a food sample with a peptide substrate. WO 2018-224561 defines food spoilage as “an unsatisfactory change in the sensory characteristics of a food stuff”. WO 2018-224561 relates to a different field than in vitro diagnostic methods of bodily fluids.

WO 2018-085895 discloses chemiluminescent BRET based sensors for detection of proteases in UHT and raw milk; the Examples show exclusively UHT and raw milk. The proteases detected are extracted from the food spoilage organism Pseudomonas fluorescens. WO 2018-085895 relates to the field of food spoilage, in particular a biosensor for detection of Pseudomonas fluorescens in milk. Moreover, WO 2018-085895 relates to a different field than in vitro diagnostic methods of bodily fluids and teaches detection using the RLuc2/Clz400a/GFP2 system at wavelengths 410/515 nm.

There is a need for a rapid, in vitro diagnostic method that identifies infection in bodily fluid samples and that can be carried out preferably be carried out at the point of care. In addition, there is a need for a rapid, in vitro diagnostic method that can be carried out in without surface effects.

There is also a rapid, in vitro diagnostic method that can be carried out in samples contaminated with blood.

In particular, there is also a need for a rapid, diagnostic method that can identify the presence of periprosthetic joint infection, peritoneal dialysis-related peritonitis and/or cerebrospinal associated infection in a sample of synovial fluid, peritoneal fluid or cerebrospinal fluid sample in the acute phase following surgery.

### SUMMARY OF THE INVENTION

The present invention provides a method for in vitro diagnosing infection in bodily fluid samples using a reagent having the general formula [a]−[b]−[c] (I), wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of bodily fluid, preferably human bodily fluid, with the reagent in a sample receptacle, and
ii) monitoring the fluorescence emission in the range of 650-900 nm from the sample in step i), wherein an increase in fluorescence emission in the range of 650-900 nm is indicative for the presence of infection in the bodily fluid sample.

According to the method is defined herein, rapid in vitro diagnosis of infection in bodily fluid samples is made possible by analysing the fluorescence emission in the range of 650-900 nm from the reagent in the presence of a samples of bodily fluid. When the target bacterial biomarker is present in the bodily fluid, the bacterial biomarker recognises and cleaves the cleavage site in the reagent, increasing the distance between the fluorescent and non-fluorescent agent, and as a result a fluorescent signal is emitted, which can be detected by a suitable detector.

Surprisingly, it has been found that the present method can be carried out directly in a bodily fluid sample without the need to enrich the bacterial cell count by, for example, a bacterial enrichment step. As a result, the present method is able to detect bacterial infection rapidly, for example, within 30 minutes, preferably within 15 minutes, of contacting the reagent with sample.

A further advantage of the present method is that the method can be run directly in the sample, that is without a sample clean-up step, as the presence of components such as red blood cells which interfere with UV wavelength based detection methods, do not cause interference with the present method.

In a second aspect, there is provided a lyophilized bead comprising a carbohydrate and a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker.

In a third aspect there is provided the use in an enzymatic assay of a lyophilized bead comprising a carbohydrate and a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, wherein the cleavage site is specific for a biomarker, preferably a bacterial biomarker.

In a fourth aspect there is provided a method for manufacturing a lyophilized bead comprising a carbohydrate and a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
said method comprising the steps of:
   i) mixing an aqueous solution of the reagent with a carbohydrate to provide an aqueous solution of the reagent comprising a carbohydrate
   ii) freeze-drying the aqueous solution of the reagent comprising a carbohydrate to provide a dried bead comprising the reagent and carbohydrate.

In a fifth aspect there is provided a kit for in vitro infection in bodily fluids, comprising:
a) a container comprising a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
b) a set of instructions for carrying out the method as described herein.
c) optionally a container containing a diluent.

In a sixth aspect, there is provided a system, comprising:\
a) a container comprising a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
b) a device adapted to receive the container and monitor the fluorescence signal emitted from the reagent in the presence of a sample of bodily fluid with the reagent, wherein the system is adapted to carry out a method as described herein.

### DESCRIPTION OF EMBODIMENTS

The term “subject” as used herein means an animal or human individual who is at risk of or suspected of having an infection. The terms “patient,” “subject,” “individual,” and the like are used interchangeably herein, and refer to any animal or human amenable to the methods described herein. In certain non-limiting embodiments, the patient, subject or individual is a human.

The term “bodily fluid sample” as used herein means a biological material isolated from a subject in the fluid state.

The term “biomarker” as used herein means an enzyme, transpeptidase, peptidase or protease that is bound to, or excreted from a cell or microorganism, The biomarker is indicative of the status of a particular analyte or subject, for example the biomarker is indicative of the presence of a particular bacteria, or is indicative of a particular clinical situation, for example the clinical situation may be an infection, disease state or metabolic state.

The term “bacterial biomarker” as used herein means a transpeptidase, peptidase or protease that is bound to, or excreted from a bacterium or group of bacteria. The bacterial biomarker is indicative for the presence of infection in a bodily fluid, preferably periprosthetic joint infection, peritoneal dialysis-related peritonitis and/or cerebrospinal associated infection.

The terms “protease” and “peptidase” as used herein mean a protein present in bacteria, secreted by bacteria or present in the membrane of bacteria capable of cleaving an amino acid motif.

The term “transpeptidase” as used herein means a protein present in the membrane of bacteria capable of cleaving an amino acid motif of a first protein and covalently linking the cleaved amino acid motif to a second protein.

The term “peptide” as used herein means a reagent comprising at least 3 amino acids. Preferably, the peptide comprises no more than 20 amino acids. The amino acids used may be any amino acid, preferably chosen from the group of naturally occurring amino acids or from the group of synthetic amino acids, in particular derivatives of natural amino acids.

The term “cleavage site” as used herein means an amino acid motif that is cleaved by a specific compound whereby the cleavage site comprises one or more amide bonds. Preferably, the cleavage site contains 3 amino acids. Preferably, the cleavage site is cleaved by the action of a protease or transpeptidase.

“Sample” or “biological sample” as used herein means a biological material isolated from an individual. The biological sample may contain any biological material suitable for detecting the desired biomarkers, and may comprise cellular and/or non-cellular material obtained from the individual.

The term “monitoring”, “measuring” “measurement,” “detecting” or “detection,” as used herein means assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

The term “infection” as used herein means a clinically relevant amount of bacteria which is indicative of a subject showing clinical signs of an infection.

The term “clinically relevant amount of bacteria” as used herein means a bacterial colony count of more than 0.1 CFU/mL, more than 1 CFU/mL, 1×10¹ CFU/mL, more than 1×10² CFU/mL, preferably more than 1×10³ CFU/mL, more preferably more than 1×10⁴ CFU/mL, even more preferably 1×10⁵ CFU/mL, yet more preferably 1×10⁶ CFU/mL, even yet more preferably 1×10⁷ CFU/mL, and even yet more preferably 1×10⁸ CFU/mL.

In a first aspect there is provided a method for in vitro diagnosing infection in bodily fluid samples using a reagent having the general formula [a]−[b]−[c] (I), wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of bodily fluid, preferably a human bodily fluid, with the reagent in a sample receptacle, and
ii) monitoring the fluorescence emission in the range of 650-900 nm from the sample in the receptacle in step i), wherein an increase in fluorescence emission in the range of 650-900 nm is indicative for the presence of infection in the bodily fluid sample.

Preferably, the bodily fluid sample is selected from the group consisting of gingival fluid, amniotic fluid, urine, serous fluid, synovial fluid, peritoneal dialysis fluid, semen, sebum and cerebrospinal fluid, preferably wherein the bodily fluid is selected from the group consisting of synovial fluid, peritoneal dialysis fluid and cerebrospinal fluid, even more preferably wherein the bodily fluid is selected from the group consisting of synovial fluid and peritoneal dialysis fluid.

Preferably, the sample is selected from the group consisting of cerebrospinal fluid, synovial fluid and peritoneal fluid.

Preferably, the bodily fluid sample is a serous fluid selected from the group consisting of pleural fluid, pericardial fluid, peritoneal fluid and combinations thereof.

In the context of the present invention, “serous fluid” means the fluid that is present in the the pleural, pericardial, and peritoneal cavities. These cavities are each lined by two membranes referred to as the serous membranes. One membrane lines the cavity wall (parietal membrane), and the other covers the organs within the cavity (visceral membrane). The fluid between the membranes is called serous fluid.

Samples of serous fluids are collected by needle aspiration from the respective cavities. These aspiration procedures are referred to as thoracentesis (pleural), pericardiocentesis (pericardial), and paracentesis (peritoneal).

It is known that serous fluids are formed as ultrafiltrates of plasma. Under normal conditions, oncotic pressure from serum proteins is the same in the capillaries on both sides of the membrane. Therefore, the hydrostatic pressure in the parietal and visceral capillaries causes fluid to enter between the membranes. The filtration of the plasma ultrafiltrate results in increased oncotic pressure in the capillaries that favours reabsorption of fluid back into the capillaries. This produces a continuous exchange of serous fluid and maintains the normal volume of fluid between the membranes. Examples of serous fluids are pleural fluid, pericardial fluid and peritoneal fluid.

Preferably, the method described herein relates to the in vitro diagnosis of infection in serous fluid.

Fluid found in the pleural cavity, located between the parietal pleural membrane lining the chest wall and the visceral pleural membrane covering the lungs, is referred to as pleural fluid. Preferably, the method described herein relates to the in vitro diagnosis of infection in pleural fluid, more preferably the method described herein relates to the in vitro diagnosis of bacterial pneumonia and/or tuberculosis.

Pericardial fluid is found between the pericardial serous membranes. The pericardial fluid may become infected. Preferably, the method described herein relates to the in vitro diagnosis of infection in pericardial fluid, more preferably the diagnosis of bacterial endocarditis and pericarditis.

Fluid between the peritoneal membranes is called ascites, and the fluid is commonly referred to as ascitic fluid or peritoneal fluid. Preferably, the method described herein relates to the in vitro diagnosis of infection in ascetic fluid (peritoneal fluid), more preferably the diagnosis of peritonitis.

In some circumstances, peritoneal fluid is provided by peritoneal washing or lavage by conventional transabdominal techniques readily familiar to the physician.

Within the context of the present application, “peritoneal dialysis fluid” means the fluid that has been infused into the peritoneal (abdominal) cavity as part of the technique known as continuous ambulatory peritoneal dialysis (CAPD) or continuous cycling peritoneal dialysis (CCPD; also known as automated peritoneal dialysis (APD) or another other similar technique. In these techniques, metabolic waste products and excess electrolytes and other materials are transferred from the body into a fluid infused into the peritoneal cavity, using the peritoneal membrane as a dialyzing membrane, over a period of time commonly referred to as the dwell time. The fluid for dialysis is introduced into the peritoneal cavity by a transabdominal connection. By virtue of the introduction of this artificial connection to the peritoneal cavity, these patients are exposed to an increased risk or peritoneal inflammation or peritoneal infection, commonly referred to as peritonitis.

Preferably, the method described herein relates to the in vitro diagnosis of infection in peritoneal dialysis fluid, more preferably the diagnosis of peritonitis associated with peritoneal dialysis.

The peritoneal dialysis fluid sample can be provided by any convenient means. If the patient is a CAPD patient, a sample of dialysate fluid can be taken as the waste (spent) fluid is removed from the peritoneal cavity. Alternatively, the peritoneal dialysis fluid sample can be provided by removing a sample via the sample port of the waste bag for collecting the peritoneal dialysis fluid after the stipulated dwell time has passed. Obtaining samples from such waste bags can be done using standard methods.

Within the context of the present application “Synovial fluid” means the biological fluid that is found in the synovial cavity of a joints between the cartilage and synovium of facing articulating surfaces. It is known that synovial fluid provides nourishment to the cartilage and also serves as a lubricant for the joints. The cells of the cartilage and synovium secrete fluid and the fluid lubricates and reduces friction between the articulating surfaces.

Preferably, synovial fluid is from a human or animal subject, more preferably from a human subject.

A typical human synovial fluid comprises approximately 85% water in addition to dissolved proteins, glucose, mineral ions, hormones, etc. The proteins, albumin and globulins, are typically present in synovial fluid and are believed to play an important role in the lubrication of the joint area. Other proteins are also found in human synovial fluid, including the glycoproteins such as alpha-1-acid glycoprotein (AGP), alpha-1-antitrypsin (A1AT) and lubricin. Another compound that is present in human synovial fluid is hyaluronic acid. Hyaluronic acid is also believed to play a role in lubrication. Human synovial fluid further includes other compounds, such as polysaccharides and phospholipids. The phospholipid, dipalmitoylphosphatidylcholine (DPPC), is also present in human synovial fluid. DPPC is generally regarded as surfactant and is also believed to play a role in the lubrication of the joint.

Bacterial contamination of synovial fluid can lead to sceptic arthritis. Preferably, the method described herein relates to the in vitro diagnosis of infection in synovial fluid, more preferably the diagnosis of sceptic arthritis.

In the case of bacterial contamination of synovial fluid in the presence of a joint replacement, this can lead to periprosthetic joint infection. Preferably, the method described herein relates to the in vitro diagnosis of infection in synovial fluid (peritoneal fluid), more preferably the diagnosis of peritonitis. periprosthetic joint infection (PJI).

Within the context of the present invention, “semen” refers to the section of male reproductive organs. An infection associated with semen is bacterial prostatis. Preferably, the method described herein relates to the in vitro diagnosis of infection in semen, more preferably the diagnosis of bacterial prostatis.

Amniotic fluid is the fluid that surrounds the foetus. An infection associated with amniotic fluid is intra-Amniotic infection. Preferably, the method described herein relates to the in vitro diagnosis of infection in amniotic fluid, more preferably the diagnosis of intra-Amniotic infection.

Urine is a liquid by-product of metabolism in humans and in many other animals. Urine flows from the kidneys through the ureters to the urinary bladder. Urination results in urine being excreted from the body through the urethra. Bacterial infection in urine is typically referred to as a urinary tract infection (UTI). Preferably, the method described herein relates to the in vitro diagnosis of infection in urine, more preferably the diagnosis of urinary tract infections.

Cerebrospinal fluid is the clear watery fluid which fills the space between the arachnoid membrane and the pia mater. Preferably, the method described herein relates to the in vitro diagnosis of infection in cerebrospinal fluid.

Preferably, the bodily fluid is gingival fluid. Preferably, the method disclosed herein relates to the in vitro diagnosis of gingivitis.

Preferably, there is provided a method for in vitro diagnosing periprosthetic joint infection, peritoneal dialysis related peritonitis and/or cerebrospinal associated infection using a reagent having the general formula [a]−[b]−[c] (I)
wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of synovial fluid, peritoneal fluid or cerebrospinal fluid with the reagent in a sample receptacle, and
ii) monitoring the fluorescence emission in the range of 650-900 nm from the sample in step i), wherein an increase in fluorescence emission in the range of 650-900 nm is indicative for the presence of periprosthetic joint infection, peritoneal dialysis-related peritonitis and/or cerebrospinal associated infection.

Accordingly, there is provided a method for in vitro diagnosing peritoneal dialysis-related peritonitis in a subject using a reagent having the general formula [a]−[b]−[c] (I) wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of peritoneal fluid with the reagent a sample receptacle, and
ii) monitoring the fluorescence emission in the range of 650-900 nm from the sample in step i), wherein an increase in fluorescence emission in the range of 650-900 nm is indicative for the presence of peritoneal dialysis-related peritonitis.

Yet another advantage of the method described herein is that peritoneal dialysis-related peritonitis is diagnosed by monitoring the fluorescence emission in the range of 650-900 nm from the sample of peritoneal dialysis fluid, enabling the method to be carried out directly on the collected peritoneal dialysis fluid after the necessary dwell time.

The advantage associated with the fluorescence emission being generated by cleavage of the cleavage site by a bacterial biomarker, is that the measured fluorescence emission is indicative of bacterial infection. In current clinical practice, the standard way for a patient or healthcare professional to check for bacterial presence is by observing whether the dialysis waste fluid is cloudy. However, the so-called cloudy bag test is prone to false positive results as a cloudy dialysis waste fluid may also be caused by elevated levels of either cellular or non-cellular constituents in the peritoneal dialysis fluid, such as elevated leucocyte and red blood cell counts. Consequently, the method described herein is able to distinguish between a bacterial infection (peritonitis) and inflammatory (non-infectious) disease states associated with peritoneal dialysis, as the fluorescence emission in the range of 650-900 nm is indicative of the presence of a bacterial biomarker.

Accordingly, there is provided a method for in vitro diagnosing of periprosthetic joint infection in a subject using a reagent having the general formula [a]−[b]−[c] (I) wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of synovial fluid a sample receptacle, and
ii) monitoring the fluorescence emission in the range of 650-900 nm from the sample in step i), wherein an increase in fluorescence emission in the range of 650-900 nm is indicative for the presence of periprosthetic joint infection.

Yet another advantage of the method described herein is that the periprosthetic joint infection can be diagnosed by monitoring the fluorescence emission in the range of 650-900 nm from the sample of synovial fluid, enabling the method to be carried out directly on the collected synovial fluid.

The advantage associated with the fluorescence emission being generated by cleavage of the cleavage site by a bacterial biomarker, is that the measured fluorescence emission is indicative of bacterial infection. State of the art methods to detect PJI typically detect elevated inflammatory biomarkers associated with bacterial infection. However, such methods based on inflammatory biomarkers cannot be used in the period immediately after a traumatic event or operation as the inflammatory biomarkers are elevated in this period. Consequently, methods based on inflammatory biomarkers are prone to false positive results. The method described herein is able to distinguish between a bacterial infection (PJI) and an inflammatory to trauma or an operation (e.g. joint replacement), as the fluorescence emission in the range of 650-900 nm is indicative of the presence of a bacterial biomarker. Consequently, the method described herein can be carried out within 8 weeks, preferably 6 weeks, preferably 4 weeks, preferably 2 weeks, preferably 1 week after a trauma or operation.

Accordingly, there is provided a method for in vitro diagnosing cerebrospinal associated infection in a subject using a reagent having the general formula [a]−[b]−[c] (I) wherein:
[a] is a fluorescent agent having an emission wavelength of 650-900 nm,
[c] is a non-fluorescent agent having an absorption wavelength of 650-900 nm, for quenching said emission of said fluorescent agent,
[b] is a peptide comprising a cleavage site, the cleavage site being specific for a bacterial biomarker, wherein the method comprises the steps of:
i) contacting a sample of cerebrospinal fluid a sample receptacle, and
ii) monitoring the fluorescence emission in the range of 650-900 nm from the sample in step i), wherein an increase in fluorescence emission in the range of 650-900 nm is indicative for the presence of cerebrospinal associated infection.
iii) Yet another advantage of the method described herein is that the infection in CSF can be diagnosed by monitoring the fluorescence emission in the range of 650-900 nm from the sample of synovial fluid, enabling the method to be carried out directly on the collected CSF.\
iv) The advantage associated with the fluorescence emission being generated by cleavage of the cleavage site by a bacterial biomarker, is that the measured fluorescence emission is indicative of bacterial infection. State of the art methods to detect infection in CSF typically detect elevated inflammatory biomarkers associated with bacterial infection. However, such methods based on inflammatory biomarkers cannot be used in the period immediately after a traumatic event or operation as the inflammatory biomarkers are elevated in this period. Consequently, methods based on inflammatory biomarkers are prone to false positive results. The method described herein is able to distinguish between a bacterial infection and an inflammatory to trauma or an operation (e.g. placement of spinal implant), as the fluorescence emission in the range of 650-900 nm is indicative of the presence of a bacterial biomarker. Consequently, the method described herein can be carried out within 8 weeks, preferably 6 weeks, preferably 4 weeks, preferably 2 weeks, preferably 1 week after a trauma or operation.

Yet another advantage of the method described herein is that the periprosthetic joint infection, by monitoring the fluorescence emission in the range of 650-900 nm from the sample of synovial fluid, enabling the method to be carried out directly on the collected synovial fluid.

Preferably, the bodily fluid sample comprises at least 70 wt. % water, more preferably at least 85% water, based on dry weight of the sample. Preferably, the bodily fluid sample comprises at least 70 wt. % and protein. Preferably, the bodily fluid comprises least 70 wt. % and protein, wherein if whey and casein proteins are present, these are present in a ratio in the range of 90:10 to 50:50. Preferably, the whey:casein ratio is at least 25:75, preferably at least 30:70, more preferably at least 40:60.

Preferably, step ii) comprises monitoring the fluorescence emission in the range of 700-850 nm, more preferably in the range of 750-850 nm. Preferably, an increase in fluorescence emission in the range of 700-850 nm, more preferably in the range of 750-850 nm is indicative infection.

Preferably, an increase in fluorescence emission in the range of 700-850 nm, more preferably in the range of 750-850 nm is indicative for periprosthetic joint infection, peritoneal dialysis-related peritonitis and/or cerebrospinal associated infection.

The fluorescent agent having an emission wavelength of 650-900 nm is preferably a cyanine moiety (dye). Preferably, the non-fluorescent agent having an emission wavelength of 650-900 nm is a cyanine moiety (dye).

Preferably, the fluorescent agent having an emission wavelength of 650-900 nm comprises a cyanine moiety, preferably a sulfo-cyanine moiety and wherein the non-fluorescent agent having an absorption wavelength of 650-900 nm comprises a cyanine moiety, preferably a sulfo-cyanine moiety

Preferably, the fluorescent agent is a cyanine dye having an emission wavelength of 650-900 nm and the non-fluorescent agent is a cyanine dye having an absorption wavelength of 650-900 nm.

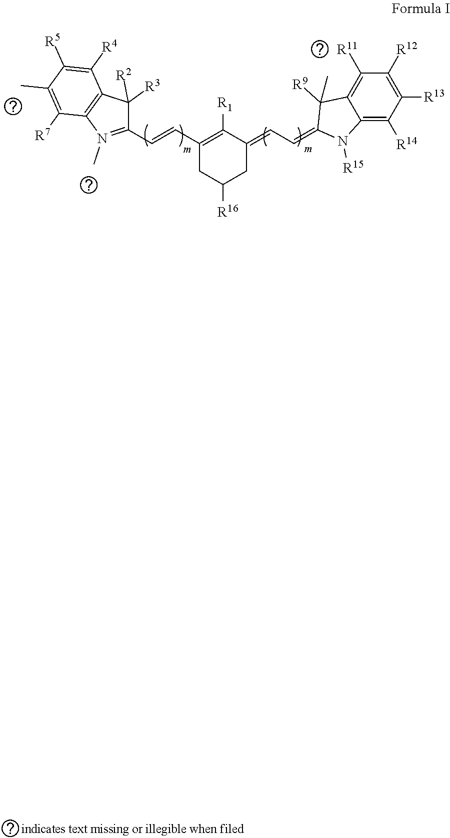

In an embodiment, the first fluorescent agent is a cyanine dye having the general formula as shown in formula I, wherein R¹ is selected from the group consisting of H, halo, substituted phenyl (R₁₈-Ph-), wherein R₁₈ comprises a functional group (FG) that does not directly react with carboxyl, hydroxyl, amino or thiol groups, and

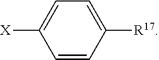

Preferably, R₁₈ is (CH₂)_{q}R₁₉FG moiety, wherein R₁₉ is a optionally substituted hydrocarbyl or amidyl moiety and FG is a functional group that does not directly react with carboxyl, hydroxyl, amino or thiol groups and q is an integer between 0 and 6, preferably between 1 and 5, more preferably between 2 and 4. R₁₉ is preferably selected from the group consisting of (CH₂)₂CONH(CH₂)₂CONHFG wherein FG is a functionality selected from the group —SO₃H, triazine, cylcooctyne, azide, alkyne, tetrazine, alkene, alkene and tetrazole. Preferably, R₁₈FG is CH₂)₂CONH(CH₂)₂CONH-dibenzocyclooctyne.

X is selected from the group consisting of O, S, NH and N-hydrocarbyl; where R¹⁷ is selected from the group consisting of carboxyl, amino and sulfanato; R², R³, R⁹,R¹⁰ are each independently selected from the group consisting of H and hydrocarbyl; R⁴, R⁵, R¹¹, R¹² are each independently selected from the group consisting of H, hydrocarbyl and sulfanato or together with the atoms to which they are bonded form an aromatic ring; R⁶, R⁷, R¹³, R¹⁴ are each independently selected from the group consisting of H and hydrocarbyl, R⁸ and R¹⁵ are each independently selected from the group consisting of hydrocarbyl, (CH₂)_{q}FG or (CH₂)PLN wherein at least one of R⁸ and R¹⁵ is (CH₂)_{q}FG, wherein q is an integer from 1 to 20 and FG is a functional group that does not directly react with carboxyl, hydroxyl, amino or thiol groups, wherein p is an integer from 1 to 20 and LN is a linker group that reacts with carboxyl, hydroxyl, amino or thiol groups; R¹⁶ is H or hydrocarbyl. Preferably, the functional group comprises a functionality selected from the group —SO₃H, triazine, cylcooctyne, azide, alkyne, tetrazine, alkene, alkene and tetrazole. Preferably the linker is selected from the group consisting of mercapto, amino, haloalkyl, phosphoramidityl, N-hydroxy succinidyl ester, sulfo N-hydroxysuccinimidyl ester, isothiocyanato, iodoacetamidyl, maleimidyl and an activated carboxylic acid.

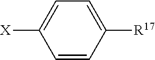

Preferably, the fluorescent agent is an agent wherein R¹ is wherein X is 0 and R¹⁷ is SO₃Na; R², R³, R⁹,R¹⁰ are hydrocarbyl, preferably methyl; R⁴ and R¹¹ are H and R⁵ and R¹² are H or sulfanato; R⁶, R⁷, R¹³, R¹⁴ are H; R₈ is (CH₂)_{q}FG where q is 4 and FG is sulfanato; R¹⁵ is (CH₂)PLN where p is 5 and LN is carboxyl, R¹⁶ is H.

Even more preferably, the fluorescent agent is an agent wherein R¹ is

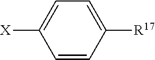

wherein X is O and R¹⁷ is SO₃Na; R², R³, R⁶, R¹⁰ are methyl; R⁴ and R¹¹

are H and R⁵ and R¹² are sulfanato; R⁶, R⁷, R¹³, R¹⁴ are H; R⁸ is (CH₂)_{q}FG where q is 4 and FG is sulfanato; R¹⁵ is (CH₂)PLN where p is 5 and LN is carboxyl, R¹⁶ is H.

Preferably, the fluorescent agent is an agent corresponding to formula II.

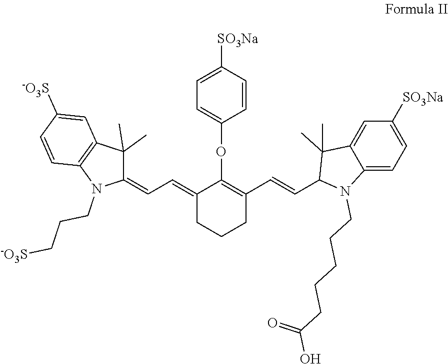

Most preferably, the fluorescent agent is an agent wherein R₁ is R₁₈ph, where R₁₈ is a N-[3-(11,12-didehydrodibenzo[b,f]azocin-5(6H)-yl)-3-oxopropyl]propanyl moiety, R₁₆ is H, R₄ and R₁₁ are H, R₅ and R₁₂ is SO₃H, R¹² is (CH₂)₃SO₃H, R⁶, R⁷, R¹³, R¹⁴ are H as shown in Formula V.

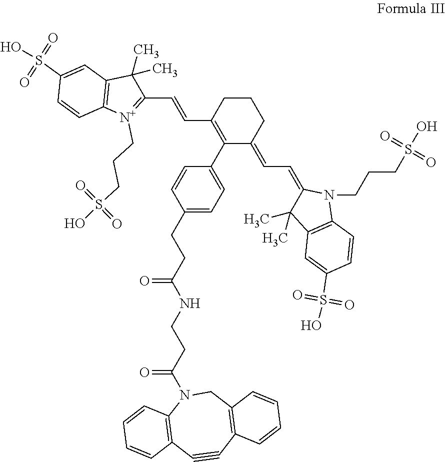 [SMILES: CC1(C)C(/C=C/C2=C(c3ccc(CCC(=O)NCCC(=O)N4Cc5ccccc5C#Cc5ccccc54)cc3)C(=C/C=C3/N(CCCS(=O)(=O)O)c4ccc(S(=O)(=O)O)cc4C3(C)C)/CCC2)=[N+](CCCS(=O)(=O)O)c2ccc(S(=O)(=O)O)cc21]

Preferably, the fluorescent agent comprises a sulfo-cyanine moiety.

The non-fluorescent agent having an absorption wavelength of 650-900 nm, is a compound that has little or no intrinsic fluorescence and which can efficiently quench the fluorescence from a proximate fluorophore with little background. In an embodiment the non-fluorescent agent is a cyanine molecule. Cyanine molecules, also referred to as cyanine dyes, include compounds having two substituted or unsubstituted nitrogen-containing heterocyclic rings joined by a polymethine chain.

Preferably, the non-fluorescent agent comprises a sulfo-cyanine moiety.

In a preferred embodiment, the non-fluorescent agent is an agent wherein R¹ is chloro, R², R³, R⁹⁷R¹⁰ are methyl; R⁴ is H and R⁵ is N-hydrocarbyl, preferably N[(CH₂)₃SO₃Na]₂;
R¹¹ and R¹² form a aromatic ring monosubsituted with sulfanato group; R⁶, R⁷, R¹³, R¹⁴ are
H; R⁸ is (CH₂)_{q}FG where q is 3 and FG is sulfanato; R¹⁵ is (CH₂)PLN where p is 5 and LN is carboxyl; R¹⁶ is H.

In another embodiment, the fluorescent agent and the non-fluorescent are the same agent, preferably wherein R¹ is

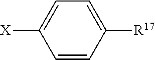

wherein X is O and R¹⁷ is SO₃Na, R², R³, R⁹,R¹⁰ are hydrocarbyl, preferably methyl, R⁴, R⁵, R¹¹, R¹² are H, R⁶, R⁷, R¹³, R¹⁴ are H, R⁸ is (CH₂)_{q}FG, where q is 4 and FG is sulfanato, R¹⁵ is (CH₂)PLN where p is 5 and LN is carboxyl, R¹⁶ is H.

The non-fluorescent agent may also be a quenching moiety for example BHQ3 (Biosearch), QC-1 (Li-COR.com), or particles comprising such compounds, for example gold nanoparticles and ferro-nanoparticles. In an embodiment, the reagent is a nanoparticle comprising a peptide as defined herein.

Examples of fluorescent agents that can be used with in present invention include, but are not limited to, Alexa Fluor® 660, Alexa Fluor® 680, Alexa Fluor® 700, Alexa 25 Fluor® 750 ATTO 680, ATTO 700, DY-647, DY-650, DY-673, DY-675, DY-676, DY-680, DY-681 DY-682, DY-690, DY-700, DY-701 DY-730, DY-731 DY-732, DY734, DY-750, DY-751 DY-752, DY-776, DY-781 DY-782, DY-831, La Jolla Blue, Cy5, Cy5.5, Cy7, IRDye® 800CW, IRDye® 38, IRDye® 800RS, IRDye® 700DX, IRDye® 680, TF7WS, TF8WS, TideDye or TideQuencher, among others. “Alexa Fluor” dyes are available from Molecular Peptides Inc., Eugene, OR, U.S.A. (www.peptides.com). “ATTO” dyes are available from ATTO-tec GmbH, Siegen, Germany (www.atto-tec.com). “Dr dyes are available from Dyomics GmbH, Jena, Germany (www.dyomics.com). La Jolla Blue is available from Hyperion Inc. “Cy” dyes are available from Amersham Biosciences, Piscataway, NJ, U.S.A. (www.amersham.com). “IRDye® infrared dyes” are available from LI-COR® Bioscience, Inc. Lincoln, N E, U.S. A (www.licor.com).

Preferably, the fluorescent agent is selected from the group consisting of Cy7, Cy7.5, TF7WS, TF8WS and IRDye800CW.

Preferably, the non-fluorescent agent having an emission wavelength of 650-900 nm is a cyanine moiety, preferably QC-1.

Most preferably, the reagent comprises a fluorescent agent selected from the group consisting of Cy7, Cy7.5, TQ7WS and IRDye800CW and a non-fluorescent agent having an emission wavelength of 650-900 nm being QC-1.

The reagent preferably comprises a fluorescent agent selected from the group consisting of Cy7, Cy7.5, TF7WS and IRDye800CW and a non-fluorescent agent having an emission wavelength of 650-900 nm being QC-1 and a cleavage site comprising at least three amino acids.

The bodily fluid sample is a human or animal bodily fluid sample, more preferably the bodily fluid is a human bodily fluid.

Preferably, the sample has a volume in the range of 10 μl to 3000 μL, more preferably, 50 μL to 2500 μL, even more preferably 100 to 2000 μL, yet more preferably 150 to 1500 μL and most preferably 200 to 1000 μL.

Preferably, the concentration of reagent in the sample receptacle after addition of the sample, and optionally a diluent, is in the range of 0.01 to 10 uM, preferably 0.02 to 8 uM, more preferably 0.05 to 5 uM, even more preferably 0.1 to 2.5 uM.

Preferably, step i) comprises the step of adding a diluent. Preferably, the diluent is an aqueous solution.

Preferably wherein the diluent comprises a buffering agent selected from the group consisting of MOPS, phosphate, citrate, HEPES TRIS-HCl, phosphate buffered saline. The preparation of aqueous solutions comprising buffering agents is known to the skilled person.

Preferably the diluent has a pH in the range of 5-9, more preferably in the range 5.5 to 8.5, even more preferably 5.75 to 8.25, yet more preferably 6 to 8.

Preferably, the volume ratio of diluent to sample is in the range of 0.1 to 100, more preferably 0.5 to 75, even more preferably in the range 1 to 50.

Preferably the sample is diluted in an assay buffer, preferably wherein the sample is diluted by a factor in the range of 1:10 to 1:10000, more preferably 1:100 to 1:1000.

Preferably, the assay buffer is capable of maintaining a pH in the range of 5-9, preferably about 6 to about 8, more preferably about 6.5 to about 7.5. preferably the buffer comprises HEPES, PIPES, Tris-Hydrochloride (Tris-HCl), phosphate, phosphate buffered saline, or MOPS. Preferably the buffer is selected from the group of phosphate buffer and phosphate buffered saline. Preferably the phosphate buffer comprises sodium and/or potassium ions.

Preferably, the buffer contains a detergent that is capable of lysing the cellular material in the bodily fluid sample.

Preferably, the buffer comprises one or more non-ionic detergents, selected from the group consisting of N-octyl- -D-glucopyranside, N-octyl-D-maltoside, ZWITTERGENT 3.14, deoxycholate; n-Dodecanoylsucrose; n-Dodecyl- -Dglucopyranoside; n-Dodecyl- -D-maltoside; n-Octyl- -D-glucopyranoside; n-Octyl-p-Dmaltopyranoside; n-Octyl-p-D-thioglucopyranoside; n-Decanoylsucrose; n-Decyl-p-D-maltopyranoside; n-Decyl-p-D-thiomaltoside; n-Heptyl- -D-glucopyranoside; n-Heptyl-p-D-thioglucopyranoside; n-Hexyl- -D-glucopyranoside; n-Nonyl-p-D-glucopyranoside; n-Octanoylsucrose; n-Octyl- -D-glucopyranoside; n-Undecyl- -D-maltoside; APO-10; APO12; Big CHAP; Big CHAP, Deoxy; BRIJ® 35; d2E5; d2E6; Ci2E8; Ci2E9; Cyclohexyl-methyl-p-D-maltoside; Cyclohexyl-n-hexyl-p-D-maltoside; Cyclohexyl-n-methyl- -D-maltoside; Digitonin; ELUGENT™; GENAPOL® C-100; GENAPOL® X-080; GENAPOL® X-100; HECAMEG; MEGA-10; MEGA-8; MEGA-9; NOGA; NP-40; PLURONIC® F-127; TRITON® X-100; TRITON® X-I 14; TWEEN® 20; or TWEEN® 80 and mixtures thereof.

The buffer preferably comprises an ionic detergent selected from the group consisting of BATC, Cetyltrimethylammonium Bromide, Chenodeoxycholic Acid, Cholic Acid, Deoxycholic Acid, Glycocholic Acid, Glycodeoxycholic Acid, Glycolithocholic Acid, Lauroylsarcosine, Taurochenodeoxycholic Acid, Taurocholic Acid, Taurodehydrocholic Acid, Taurolithocholic Acid, Tauroursodeoxycholic Acid, TOPPA and mixtures thereof.

Preferably, the buffer comprises a zwitterionic detergent selected from the group consisting of amidosulfobetaines, CHAPS, CHAPSO, carboxybetaines, and methylbetaines.

Preferably, the buffer comprises an anionic detergent selected from group consisting of SDS, N-lauryl sarcosine, sodium deoxycholate, alkyl-aryl sulphonates, long chain (fatty) alcohol sulphates, olefine sulphates and sulphonates, alpha olefine sulphates and sulphonates, sulphated monoglycerides, sulphated ethers, sulphosuccinates, alkane sulphonates, phosphate esters, alkyl isethionates, sucrose esters and mixtures thereof.

In the present application, a buffering agent is a chemical species that is a capable of adjusting the pH of the buffer and/or sample combination.

Preferably, the sample of bodily fluid, is contacted with the reagent directly. The term “directly” as used herein means that the sample is not subjected to a centrifugation step or other sample clean up step prior to contact with the reagent.

Preferably, the sample of bodily fluid, is contacted with the reagent directly.

Optionally, the method comprises the step of centrifuging the sample can be centrifuged to separate aggregated bacteria matter and/or precipitated biological material from a supernatant. Preferably, centrifugation can be at a force in the range of 1,000 g to 25,000 g, preferably, in the range of 3,000 g to 20,000 g, more preferably in the range of 3,700 g to 18,000 g. The centrifugation time is preferably in the range of 1 minute to 30 minutes, in the range of 2 to 20 minutes, more preferably in the range of 3 to 10 minutes. The supernatant or the aggregated bacterial matter cell resulting from centrifugation is preferably contacted with the reagent. In a preferred embodiment, the supernatant from centrifugation is preferably contacted with the reagent. In another preferred embodiment, the aggregated bacterial matter cell resulting from centrifugation is preferably contacted with the reagent.

Preferably, the method comprises the step of centrifuging the sample prior to contacting the sample with the reagent. For example, if there is some contamination of the sample with blood, it is desirable to centrifuge the sample prior to processing in the assay.

Optionally, after addition of the reagent to the sample the mixture is agitated. The mixture is preferably agitated by aspiration with a pipette, vortexing or shaking.

Preferably, the reagent is a peptide, protein or nanoparticle. Preferably, the reagent is a peptide. The peptide may be linked via a linker to a protein, nanoparticle or magnetic bead. The peptide preferably comprises between 3 and 10 amino acids, more preferably between 4 and 9 amino acids, even more preferably between 5 and 8 amino acids.

Preferably [b] comprises between 3 and 8 amino acids, more preferably between 3 and 7 amino acids, even more preferably between 3 and 6 amino acids.

The cleavage site preferably comprises of a limited number of amino acids, for example at least two, preferably at least three amino acids, more preferably at least four amino acids, even more preferably at least five amino acids.

Preferably, the cleavage site consists of between 2 and 5 amino acids, more preferably between 3 and 5 amino acids.

Preferably, [b] has the general formula Xₐₐ₁,Xₐₐ₂,Xₐₐ₃. In other words, the cleavage site preferably comprises three amino acids Xₐₐ₁,Xₐₐ₂,Xₐₐ₃ wherein
Xₐₐ₁ is preferably a small, hydrophobic amino acid, more preferably Xₐₐ₁ is selected form the group consisting of alanine, glycine, leucine, valine, norleucine, norvaline, isoleucine, isovaline, alloisoleucine;
Xₐₐ₂ is preferably a small or aromatic hydrophobic amino acid, preferably Xₐₐ₂ is selected from the group consisting of alanine, glycine, leucine, valine, norleucine, norvaline, isoleucine, isovaline, alloisoleucine and phenylalanine;
Xₐₐ₃ is preferably selected form the group consisting of alanine, glycine, leucine, valine, norleucine, norvaline, isoleucine, isovaline, alloisoleucine, more preferably Xₐₐ₃ is selected form the group consisting of alanine, glycine, leucine, valine.

In certain preferred embodiments, Xₐₐ₁ is selected form the group consisting of alanine, valine, norleucine, norvaline, isoleucine, isovaline, alloisoleucine, Xₐₐ₂ is selected from the group consisting of alanine, leucine, valine, norleucine, norvaline, isoleucine, isovaline, alloisoleucine and phenylalanine; and Xₐₐ₃ is selected form the group consisting of alanine, leucine, valine.

Preferably, the cleavage site comprises three amino acids Xₐₐ₁,Xₐₐ₂,Xₐₐ₃ wherein
Xₐₐ₁ is preferably a small, hydrophobic amino acid, more preferably Xₐₐ₁ is selected form the group consisting of alanine, glycine, leucine, valine, norleucine, norvaline, isoleucine, isovaline, alloisoleucine; more preferably alanine, leucine, valine, norleucine, norvaline, isoleucine, isovaline, alloisoleucine;
Xₐₐ₂ is preferably a small or aromatic hydrophobic amino acid, preferably, Xₐₐ₂ is selected from the group consisting of alanine, glycine, leucine, valine, norleucine, norvaline, isoleucine, isovaline, alloisoleucine and phenylalanine; more preferably, Xₐₐ₂ is selected from the group consisting of alanine, leucine, valine, norleucine, norvaline, isoleucine, isovaline, alloisoleucine and phenylalanine;
Xₐₐ₃ is preferably selected form the group consisting of alanine, leucine, valine, norleucine, norvaline, isoleucine, isovaline, alloisoleucine, more preferably Xₐₐ₃ is selected form the group consisting of alanine, glycine, leucine, valine,
wherein the cleavage site is cleaved by a bacterial biomarker wherein the bacterial biomarker is produced by a bacteria selected from the group consisting of Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, and Candida albicans, more preferably the causative agent (bacteria) is selected from the group consisting of Bacillus spp, Staphylococcus spp, Streptococcus spp, Pseudomonas spp, Escherichia coli and combinations thereof, most preferably from the group of Bacillus spp and, Pseudomonas spp

Preferably, the cleavage site comprises three amino acids Xₐₐ₁,Xₐₐ₂,Xₐₐ₃ wherein
Xₐₐ₁ is preferably a small, hydrophobic amino acid, more preferably Xₐₐ₁ is selected form the group consisting of alanine, glycine, leucine, valine, norleucine, norvaline, isoleucine, isovaline, alloisoleucine; more preferably alanine, leucine, valine, norleucine, norvaline, isoleucine, isovaline, alloisoleucine;
Xₐₐ₂ is preferably a charged amino acid, preferably, Xₐₐ₂ is selected from the group consisting of aspartic acid, glutamic acid, lysine, arginine,
Xₐₐ₃ is preferably selected form the group consisting of alanine, leucine, valine, norleucine, norvaline, isoleucine, isovaline, alloisoleucine, more preferably Xₐₐ₃ is selected form the group consisting of alanine, glycine, leucine, valine,
wherein the cleavage site is cleaved by a bacterial biomarker wherein the bacterial biomarker is produced by bacteria selected from the group consisting of Enterococcus faecalis, Enterococcus faecium, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, and Candida albicans, more preferably the causative agent (bacteria) is selected from the group consisting of Staphylococcus spp, Streptococcus spp, Escherichia coli and combinations thereof.

Preferably, [b] comprises an amino acid sequence selected from the group consisting of AAA, ALA, AAL, LAA, FAA, AFA, AAF, FGG, GFG, GGF, GGG, LGG, GLG, GGL and GGA. More preferably, [b] comprises an amino acid sequence selected from the group consisting of FGG, GFG, GGF, GGG, LGG, GLG, GGL and GGA. Yet more preferably, [b] comprises an amino acid sequence selected from the group consisting of AAA, ALA, AAL, LAA, FAA, AFA, AAF. Even more preferably, [b] comprises an amino acid sequence selected from the group consisting of FAA, AFA and AAF.

Preferably, [b] comprises an amino acid sequence selected from the group consisting of AVA, AAV, VAA, YAA, AYA, AAY, YGG, GFG, GGY, GGV, VGG, GVG, GAG and AGG. More preferably, [b] comprises an amino acid sequence selected from the group consisting of YGG, GFG, GGY, GGV, VGG, GVG, GAG and AGG. Yet more preferably, [b] comprises an amino acid sequence selected from the group consisting of AVA, AAV, VAA, YAA, AYA and AAY.

Preferably, [b] comprises an amino acid sequence selected from the group consisting of AEA, AAE, EAA, DAA, ADA, AAD, EGG, GEG, GGE, GGD, DGG, GDG, GRG and RGG. More preferably, [b] comprises an amino acid sequence selected from the group consisting of KGG, GKG, GGK, AKA, AAK, KAA, KAA, AKA and AAK.

Preferably, the mass ratio of [b]:([a]+[c]) is at least 1:2, preferably the mass ratio of [b]:([a]+[c]) is at least 1:3, more preferably the mass ratio of [b]:([a]+[c]) is at least 1:4, even more preferably the mass ratio of [b]:([a]+[c]) is at least 1:5.

Preferably, the reagent has the general formula [a]−[linker1]n−[b]−[-linker2]m−[c], wherein n is 0, 1 or 2, m is 0, 1 or 2, [linker1] and [linker2] are independently selected from the group of an optionally substituted hydrocarbyl group and a non-proteolytic hydrocarbyl group, preferably n is 0 or 1, m is 1 and linker2 is a non-proteolytic hydrocarbyl group, more preferably n is 0, m is 1 and linker2 is a non-proteolytic hydrocarbyl group.

Preferably, wherein the linker is a non-proteolytic hydrocarbyl group.

Preferably, the linker is selected from the group consisting of beta-alaninyl, 4-aminobutyrl, 2-(aminoethoxy)acetyl, 3-(2-aminoethoxy)propanyl, 5-aminovaleryl, 6-aminohexyl, 8-amino-3,6-dioxaoctanyl and 12-amino-4,7,10-trioxadodecanyl, preferably 6-aminohexyl.

Preferably, the reagent has the general formula [a]−[linker1]n−[b]−[-linker2]−[c] (II), wherein [linker,] and [linker2] are independently selected from the group of an optionally substituted hydrocarbyl group and a non-proteolytic hydrocarbyl group. Hydrocarbyl as used herein means optionally substituted C1-C6 alkyl. Preferably, the optionally substituted C1-C6 alkyl is substituted by O, N and S.

More preferably the reagent is of the general formula [a]−[linker1]n−[b]−[linker2]m−[c], wherein [b] is an amino acid sequence selected from the group consisting AAA, ALA, AAL, LAA, FAA, AFA, AAF, FGG, GFG, GGF, GGG, LGG, GLG, GGL and GGA and n is 1 and m is 1 and [linker1] and [linker2] are independently selected from the group consisting of beta-alaninyl, 4-aminobutyrl, 2-(aminoethoxy)acetyl, 3-(2-aminoethoxy)propanyl, 5-aminovaleryl, 6-aminohexyl

More preferably the reagent is of the general formula [a]−[b]−[linker]−[c], wherein b is an amino acid sequence selected from the group consisting of AAA, ALA, AAL, LAA, FAA, AFA, AAF, FGG, GFG, GGF, GGG, LGG, GLG, GGL and GGA, n is 0, m is 1 and [linker] is selected from the group consisting of beta-alaninyl, 4-aminobutyrl, 2-(aminoethoxy)acetyl, 3-(2-aminoethoxy)propanyl, 5-aminovaleryl, 6-aminohexyl.

More preferably the reagent is of the general formula [a]−[linker]−[b]−[c], wherein b is an amino acid sequence selected from the group consisting of AAA, ALA, AAL, LAA, FAA, AFA, AAF, FGG, GFG, GGF, GGG, LGG, GLG, GGL and GGA, n is 1 and m is 0 and [linker] is selected from the group consisting of beta-alaninyl, 4-aminobutyrl, 2-(aminoethoxy)acetyl, 3-(2-aminoethoxy)propanyl, 5-aminovaleryl, 6-aminohexyl.

Preferably, the method comprises the step of contacting the sample with a first reagent and a second reagent, wherein the first reagent has the general formula [a]−[b]−[c] (I) and [b] is a peptide comprising a cleavage site (b′) and the second reagent has the formula [a]−[d]−[c] (III), wherein [d] is a peptide comprising a cleavage site (d), wherein the cleavage site d′ is different to the cleavage site b′.

An advantage of the method disclosed herein is that it enables a multiplexing approach, whereby different reagents comprising different cleavage sites can be combined in a single sample receptacle to enable detection of different bacterial biomarkers.

Preferably, wherein the bacterial biomarker is a bacterial membrane bound protease, bacterial membrane bound transpeptidase, intracellular bacterial protease or extracellular bacterial protease, more preferably the bacterial biomarker is a bacterial membrane bound transpeptidase or an extracellular bacterial protease.

Preferably, the method has a specificity for bacterial infection over host inflammation response.

Within the context of the present application “host inflammation response” means a host inflammation biomarker, for example a host inflammation protease. Within the context of the present application “specificity” for infection over host inflammation response means that ratio of fluorescence emission in the range 650-900 nm in the presence of a bacterial biomarker to the fluorescence emission in the range 650-900 nm in the presence of a host inflammation response is at least 1.05:1, preferably at least 1.5 to 1, more preferably at least 2:1, even more preferably at least 5:1, yet more preferably at least 10:1, even yet more preferably at least 25:1, most preferably at least 50:1.

Preferably the ratio of fluorescence emission in the range 650-900 nm in the presence of a bacterial biomarker to the fluorescence emission in the range 650-900 nm in the presence of a host inflammation response is in the range of 1.05:1 to 500:1, preferably 2:1 to 400:1, more preferably 5:1 to 300:1.

Preferably the ratio of fluorescence emission in the range 650-900 nm in a sample of bodily fluid that comprises a bacterial biomarker to the fluorescence emission in the range 650-900 nm in a sample of bodily fluid that does not comprise a bacterial biomarker, is in the range of 1.05:1 to 500:1, preferably 2:1 to 400:1, more preferably 5:1 to 300:1.

Preferably the ratio of fluorescence emission in the range 650-900 nm in a sample of bodily fluid that comprises a bacterial biomarker to the fluorescence emission in the range 650-900 nm in a sample of bodily fluid that does not comprise a bacterial biomarker, is in the range of 1.05:1 to 500:1, preferably 2:1 to 400:1, more preferably 5:1 to 300:1, wherein the bodily fluid sample is selected from the group consisting of the bodily fluid sample is selected from the group consisting of gingival fluid, amniotic fluid, urine, serous fluid, synovial fluid, peritoneal dialysis fluid, semen, sebum and cerebrospinal fluid, preferably wherein the bodily fluid is selected from the group consisting of synovial fluid, peritoneal dialysis fluid and cerebrospinal fluid, even more preferably wherein the bodily fluid is selected from the group consisting of synovial fluid and peritoneal dialysis fluid.

Preferably, the method has a specificity for bacterial infection of at least 70%, preferably at least 75%, more preferably at least 80%, even more preferably at least 85%, yet more preferably at least 90%, most preferably at least 95%.

Within the context of the present application, the term “specificity percentage” is determined according to the formula:

$$Specificity \% = \frac{a}{b} \times 100$$

wherein a is the number of samples measured according to the method described herein which have a fluorescence emission in the range of 650-900 nm>threshold fluorescence emission in the range 650-900 nm;
b is the number of clinically positive samples for the target condition, wherein the clinically positive samples are determined to be positive for a condition by a relevant known reference method. Typically, a reference method for the determination of infection in a bodily fluid sample is the relevant clinical guidelines known to the skilled person.

The threshold fluorescence emission is value of the fluorescence emission that has been determined to be the cut-off value for the discrimination of a positive sample from a negative sample. Typically, the threshold fluorescence is determined according to the formula

Threshold=y×LOD
where y is an integer in the range of 1 and 10, preferably y is in an integer in the range of 2 and 9, more preferably y is an integer in the range 3 and 8, even more preferably y is an integer in the range of 4 and 7; and
LOD is the limit of detection, where the LOD is determined as the lowest detectable amount of the target analyte. Determination of the LOD is well within the capabilities of the skilled person. as described by David A Armbruster and Terry Pry, Clin Biochem Rev. 2008 Aug. 29Suppl 1/S49-S52.

In certain embodiment, the threshold is determined according to the formula

Threshold=LOB+LOD
where LOB is the mean blank fluorescence emission; and
LOD is the limit of detection, where the LOD is determined as the lowest detectable amount of the target analyte. The skilled person understands that the threshold is determined for a particular bodily fluid. In other words, the skilled person understands that the reagent will have different threshold values in, for example, peritoneal dialysis fluid and synovial fluid. The skilled person can determine the threshold using known methods. Determination of the LOD is well within the capabilities of the skilled person. as described by David A Armbruster and Terry Pry, Clin Biochem Rev. 2008 Aug. 29Suppl 1/S49-S52.

In certain embodiment, the threshold is determined according to the formula

Threshold=LOB+1.645 (standard deviation low concentration sample)

where LOB is the mean blank fluorescence emission; and LOD is the limit of detection, where the LOD is determined as the lowest detectable amount of the target analyte. Determination of the LOD is well within the capabilities of the skilled person. as described by David A Armbruster and Terry Pry, Clin Biochem Rev. 2008 Aug. 29Suppl 1/S49-S52

Preferably the method has a selectivity for gram positive bacteria. Within the context of the present application “selectivity” for gram positive bacteria means that ratio of fluorescence emission in the range 650-900 nm in the presence of a bacterial biomarker from gram positive bacteria to the fluorescence emission in the range 650-900 nm in the presence of a non-gram positive inflammation biomarker is at least 1.05:1, preferably at least 1.5 to 1, more preferably at least 2:1, even more preferably at least 5:1, yet more preferably at least 10:1, even yet more preferably at least 25:1, most preferably at least 50:1.

Preferably the method has a selectivity for gram negative bacteria. Within the context of the present application “selectivity” for gram negative bacteria means that ratio of fluorescence emission in the range 650-900 nm in the presence of a bacterial biomarker from gram negative bacteria to the fluorescence emission in the range 650-900 nm in the presence of a non-gram negative inflammation biomarker is at least 1.05:1, preferably at least 1.5 to 1, more preferably at least 2:1, even more preferably at least 5:1, yet more preferably at least 10:1, even yet more preferably at least 25:1, most preferably at least 50:1.

Within the context of the present application, “specificity” and “selectivity” are used interchangeably.

Preferably, the method has a specificity for gram positive bacteria of at least 70%, preferably at least 75%, more preferably at least 80%, even more preferably at least 85%, yet more preferably at least 90%, most preferably at least 95%.

Preferably wherein the method has a specificity for gram negative bacteria of at least 70%, preferably at least 75%, more preferably at least 80%, even more preferably at least 85%, yet more preferably at least 90%, most preferably at least 95%.

Preferably, the method detects infection wherein the bacteria (causative agent) is selected from the group consisting of Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, and Candida albicans, more preferably the causative agent (bacteria) is selected from the group consisting of Bacillus spp, Staphylococcus spp, Streptococcus spp, Pseudomonas spp, Escherichia coli and combinations thereof, most preferably from the group of Bacillus spp, Staphylococcus spp, and, Pseudomonas spp. The cleavage site is preferably specific for a bacterial biomarker is selected from the group consisting of Enterococcus faecalis, Enterococcus faecium, Pseudomonas aeruginosa, Staphylococcus aureus, Staphylococcus epidermidis, Staphylococcus lugdunensis, Staphylococcus warneri, Staphylococcus capitis, Staphylococcus caprae, Streptococcus mitis, Streptococcus oralis, Streptococcus agalactiae, Streptococcus anginosus, Streptococcus gordonii, Escherichia coli, Propionibacterium acnes, Proteus mirabilis, Granulicatella adjacens, Acinetobacter baumannii, Abiotrophia defective, Corynebacterium striatum, Corynebacterium minutissimum, Parvimonas micra, Candida parapsilosis, Candida glabrata, Candida tropicalis, and Candida albicans, more preferrably the causative agent (bacteria) is selected from the group consisting of Bacillus spp, Staphylococcus spp, Streptococcus spp, Pseudomonas spp, Escherichia coli and combinations thereof, most preferably from the group of Bacillus spp, Staphylococcus spp, and, Pseudomonas spp.

The cleavage site is preferably specific for a bacterial biomarker for bacteria selected from the group consisting of Bacillus spp, Staphylococcus spp, Streptococcus spp, Pseudomonas spp, Escherichia coli and combinations thereof. The cleavage site is preferably specific for a bacterial biomarker for bacteria from the group consisting of Bacillus cereus, Staphylococcus aureus, Streptococcus pyogenes, Pseudomonas aeruginosa, Escherichia coli and combinations thereof, most preferably from the group of Bacillus spp, Staphylococcus spp, and Pseudomonas spp.

Preferably, the cleavage site is specific for a bacterial biomarker for a bacteria species selected from the group consisting of Bacillus cereus, Staphylococcus aureus, Streptococcus pyogenes, Pseudomonas aeruginosa, Escherichia coli and combinations thereof, preferably wherein the cleavage site is specific for Pseudomonas aeruginosa and Bacillus cereus, more preferably wherein the cleavage site is specific for Pseudomonas aeruginosa.

Preferably, the cleavage site is specific for a bacterial biomarker for a bacteria species selected from the group consisting of Staphylococcus aureus, Pseudomonas aeruginosa, Escherichia coli and combinations thereof, preferably wherein the cleavage site is specific for Pseudomonas aeruginosa and Staphylococcus aureus, more preferably wherein the cleavage site is specific for Staphylococcus aureus.

In a particularly preferred embodiment, the sample is not subjected to a bacterial cell enrichment step prior to contacting the sample with the reagent. By “not subjected to a bacterial cell enrichment step” means there is not a step of incubating (ex vivo) the sample at a temperature in the range of 10-37° C. for a period time, for example for a number of hours in order to increase the optical density (turbidity, measured absorbance at 600 nm) of the sample.

Preferably, the sample is not subjected to an ex vivo step of bacterial enrichment in the temperature range of 20 to 40° C. either prior to, or after, being contacted with the reagent.

The step of monitoring the increase in fluorescence in step iii) is preferably carried out in a detector adapted to receive a container comprising the sample and reagent. Preferably, the sample is contacted with the reagent in a container and the fluorescence emitted by the reagent is monitored by a handheld or benchtop fluorescence spectrometer adapted to receive the container. The detector preferably provides a readout in relative fluorescent units (RFU). The user can compare the readout to a control and a relative increase in RFU between the control and the sample is indicative of bacterial infection.

Preferably, step of monitoring the increase in fluorescence in step iii) is preferably carried out by contacting a sample with the reagent and the fluorescence emitted by the reagent is monitored by a benchtop fluorescence spectrometer, preferably a plate reader. Preferably, the sample is contacted with the reagent, preferably in a sample tube, and the mixture comprising sample and reagent transferred to a multi-well plate, preferably a 96 well plate.

The increase in fluorescence (emission) is optionally detected by use of any of the following devices: CCD cameras, video cameras, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

Preferably, the reagent is not attached to a surface. It has been found that by providing the reagent in a form that is not covalently bound to a surface, non-specific interactions are unexpectedly reduced.

Preferably, the reagent in step i) is in the form of a lyophilized bead comprising a carbohydrate.

Preferably, the bead comprises 15 to 95 wt. % by weight of the bead carbohydrate, preferably 20 to 90 wt. %, more preferably 25 to 85 wt. %, yet more preferably 30 to 80 wt. % carbohydrate, by weight of the bead carbohydrate.

Preferably, the carbohydrate is selected from the group consisting of polyol, monosaccharide, disaccharide, polysaccharide and combinations thereof.

In a second aspect, there is provided a lyophilized bead comprising a carbohydrate and a reagent having the structure [a]−[b]−[c] (I)
wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker.

It has been found that formulating the reagent as a lyophilized bead with a carbohydrate has the unexpected advantage of increasing clinical performance of the reagent. Surprisingly, the ratio of a fluorescent emission at wavelengths in the range of 650-900 nm in the presence of a bacterial biomarker to the fluorescent emission at wavelengths in the range of 650-900 nm in the absence of a bacterial biomarker increases when the reagent is in the form of a carbohydrate comprising lyophilized bead.

Without wishing to be bound by theory, it is postulated that the inclusion of a carbohydrate in the lyophilized bead minimizes hydrophobic interactions between the reagent molecules in the bead such that an improved dequenching is observed, which in turn leads to an improved ratio in the presence and absence of bacterial biomarker, and thus to improved clinical performance.

The lyophilized bead typically has a size of in the range of 1-5 mm, preferably 2 to 4 mm.

Preferably, the bead comprises 5 to 95 wt. % by weight of the bead carbohydrate, preferably 10 to 90 wt. %, more preferably 15 to 85 wt. %, yet more preferably 20 to 80 wt. % carbohydrate, by weight of the bead carbohydrate.

Preferably, the carbohydrate is selected from the group consisting of polyol, monosaccharide, disaccharide, polysaccharide and combinations thereof. The carbohydrate is preferably a polyol, monosaccharide, disaccharide or polysaccharide. The identity of the carbohydrate can be selected by the skilled person by observing the relative activity of the lyophilized bead in the method disclosed herein. In certain embodiments, preferably a combination of carbohydrates may be used.

Preferably, the polyol is selected from the group consisting of (2R,3S)-Butane-1,2,3,4-tetrol (ethyritol), 4-O-beta-D-Galactopyranosyl-D-glucitol (lactitol), (2S,3S,4S,5S)-hexane-1,2,3,4,5,6-hexol (L-mannitol), (2R,3R,4R,5R)-hexane-1,2,3,4,5,6-hexol (D-mannitol), 4-O-alpha-D-Glucopyranosyl-D-glucitol (maltitol), (2R,3R,4R,5S)-hexane-1,2,3,4,5,6-hexol (sorbitol), (2R,3r,4S)-Pentane-1,2,3,4,5-pentol (xylitol), polyethyelene glycol and combinations thereof, preferably the polyol is selected from the group consisting of (2S,3S,4S,5S)-hexane-1,2,3,4,5,6-hexol (L-mannitol), (2R,3R,4R,5R)-hexane-1,2,3,4,5,6-hexol (D-mannitol) and combinations thereof.

Preferably the polyethyelene glycol has a molecular weight in the range of 50-5000, more preferably 100-2500, even more preferably 250-1000.

Preferably, the monosaccharide is selected from the group consisting of glucose, fructose, galactose and combinations thereof. Monosaccharides have the general formula (CH₂O)ₙ

Monosaccharides are classified according to three different characteristics: the location of their carbonyl group, the number of carbon atoms they contain, and their chiral property. If the carbonyl group is an aldehyde, the monosaccharide is an aldose. If the carbonyl group is a ketone, the monosaccharide is a ketose. Monosaccharides with three carbon atoms are called trioses and these are the smallest monosaccharides, such as dihydroxyacetone and d- and l-glyceraldehyde. Those composed of four carbon atoms are called tetroses, those with five carbons are called pentoses, those of six carbons are hexoses, and so on. Other minor monosaccharides include mannose, galactose, xylose, and arabinose. The most commonly detected pentoses are arabinoses and xyloses.

Preferably the disaccharide is selected from the group consisting of sucrose, maltose, lactose, trehalose and combinations thereof. Disaccharides consist of two monosaccharide units, linked together with glycosidic bonds in the α or β, orientation. Preferably, the disaccharide is sucrose. Sucrose consists of a molecule of α-glucose and β-fructose linked together. Preferably, the disaccharide is lactose. Lactose consists of galactose and glucose linked by a β-1,4-glycosidic bond. Preferably, the dissacharide is maltose. Maltose is typically produced by partial hydrolysis of starch and consists of two glucose units linked by an α-1,4-glycosidic bond.

Preferably, the polysaccharide is selected from the group consisting of alginate, chitosan, hyaluronic acid, cellulose derivatives, dextran and combinations thereof. Without wishing to be bound by theory, the presence of a polysaccharide carbohydrate is thought to stabilize the intramolecular interactions between the reagent molecules in the lypophilzed bead such that the optimum quenching is obtained in the lyophilized bead.

Preferably, the polysaccharide is dextran. Dextran is a polymer of anhydroglucose. Typically, dextran comprises approximately 95% alpha-D-(1-6) linkages, with the remaining a(1-3) linkages accounting for the branching of dextran. Typically, the average branch length is in the range of 1 to 50, more preferably 5 to 40, even more preferably 10 to 30. Preferably, the average branch less is less than three glucose units.

Preferably, the molecular weight (MW) of dextran is in the range of 2000 to 500 million. Typically, lower MW dextrans will exhibit slightly less branching and have a more narrow range of MW distribution. Dextrans with MW greater than 10,000 typically behave as if they are highly branched. As the MW increases, dextran molecules attain greater symmetry. Dextrans with MW of 2,000 to 10,000 dextran molecules exhibit the properties of an expandable coil. At MWs below 2,000 dextran is more rod-like. Preferably the molecular weight of dextran is in the range 2000 to 20000, more preferably in the range of 3000 to 15000, even more preferably in the range of 4000 to 10000.

The MW of dextran is measured by methods well known to the skilled person, which many include but are not limited to low angle laser light scattering, size exclusion chromatography, copper-complexation, and anthrone reagent colorimetric reducing-end sugar determination and viscosity.

Preferably, step i) of the method described herein comprises contacting a sample of bodily fluid, preferably a human bodily fluid, with the reagent in a sample receptacle wherein the reagent is a lyophilized bead as described herein.

Preferably, in step i) the reagent is provided as a lyophilized bead in a tube. In some embodiments, the lyophilized bead is dissolved in a diluent to provide a reagent in a solution of diluent in a suitable receptacle and then the reagent in the solution of diluent is contacted with the sample. Preferably, the diluent is an aqueous solution. Preferably the aqueous solution comprises water and optionally one or more buffering agents and/or pH adjustment agents.

The embodiments described herein with regards to the method, apply mutatis mutandis to the embodiments described herein with regards to the lyophilized bead.

In a third aspect there is provided a method for manufacturing a lyophilized bead comprising a carbohydrate and a reagent having the structure [a]−[b]−[c] (I)
wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
said method comprising the steps of:
i) mixing an aqueous solution of the reagent with a carbohydrate to provide an aqueous solution of the reagent comprising a carbohydrate
ii) freeze-drying the aqueous solution of the reagent comprising a carbohydrate to provide a dried bead comprising the reagent and carbohydrate.

The embodiments described herein with regards to the lyophilized bead, apply mutatis mutandis to the embodiments described herein with regards to the method of manufacturing the lyophilized bead.

Preferably, the aqueous solution of the reagent comprising a carbohydrate is subjected to a step of flash-freezing prior to freeze-drying.

Preferably, the freeze-drying step ii) has a duration in the range of 24-96 hours, preferably 30 to 90 hours, more preferably 36 to 84 hours.

In a fourth aspect, there is provided the use in an enzymatic assay of a lyophilized bead comprising a carbohydrate and a reagent having the structure [a]−[b]−[c] (I)
wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, wherein the cleavage site being specific for a biomarker,

It has been found that inclusion of a carbohydrate with a reagent comprising a fluorescent agent having an emission wavelength of 650-900 nm and a non-fluorescent agent having an absorption wavelength of 650-900 nm provides unexpected benefits to enzymatic assays.

Unexpected improvement in the analytical performance of an enzymatic assay is observed when the reagent is formulated with a carbohydrate in a lyophilized bead.

In addition, unexpected improvement in the clinical performance of an enzymatic assay is observed when the reagent is formulated with a carbohydrate in a lyophilized bead.

The embodiments described herein with regards to the method, apply mutatis mutandis to the embodiments described herein with regards to the use of the lyophilized bead in an enzymatic assay.

Preferably the enzymatic assay is an assay for the detection of enzymatic activity selected from the group consisting of cell assay, clinical assay, inhibitor assay, immunoassay and microbiological assay.

In a fifth aspect, there is provided a kit for in vitro diagnosis of infection in bodily fluid samples, comprising:
a) a container comprising a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
b) a set of instructions for carrying out the method defined herein.

Preferably, the reagent and the diluent are present in two compartments of a container, preferably wherein the compartments are separated by a pressure activatable membrane.

The preferred embodiments for the method described herein apply mutatis mutandis to the kit described herein.

The preferred embodiments for the lyophilized bead described herein apply mutatis mutandis to the kit described herein.

In a sixth aspect, there is provided a kit for in vitro diagnosis of periprosthetic joint infection, peritoneal dialysis-related peritonitis and/or cerebrospinal associated infection, comprising:
a) a container comprising a reagent having the structure [a]−[b]−[c] (I) wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
b) a set of instructions for carrying out the method defined herein

The inventors have found that by providing a system that comprises a device adapted to receive a container for receiving a sample of synovial fluid, peritoneal fluid or cerebrospinal fluid and contacting the sample with the reagent defined elsewhere herein, a rapid means of identifying an infection in said sample is provided.

Preferably, the system is a point-of-care system or a patient beside system.

Preferably, the system comprises
a) a container comprising a reagent having the structure [a]−[b]−[c] (I)
wherein:
[a] is a fluorescent agent having an emission wavelength of 650-900 nm
[c] is a non-fluorescent agent having an absorption wavelength of 650-900 nm, for quenching said emission of said fluorescent agent, [b] is a peptide comprising a cleavage site, the cleavage site being specific for a bacterial biomarker,
b) a device adapted to receive the container and monitor the fluorescence signal emitted from the reagent in the presence of a sample of synovial fluid, peritoneal fluid or cerebrospinal fluid with the reagent, wherein the system is adapted to carry out a method described herein.

The preferred embodiments for the method described herein apply mutatis mutandis to the system described herein.

The present invention has been described above with reference to a number of exemplary embodiments. Modifications and alternative implementations of some parts or elements are possible, and are included in the scope of protection as defined in the appended claims.

### EXAMPLES

### Example 1

Peptide 1: Peptide cleavage site: AFA; a fluorescent agent: IRDye 800CW (Licor Bioscience, USA); non-fluorescent agent: QC-1 (Licor Bioscience, USA).

A control peptide (A) was used having the same fluorescent agent and non-fluorescent agent as (1); Peptide cleavage site: ADA.

Biological Samples

### Example 1.1

Composition of model synovial fluid:
2 mg/mL hyaluronic acid
25 mg/mL bovine serum albumin
4 mg/mL nutrient broth powder (Difco no. 234000)
Prepared in phosphate buffered saline, pH 7.1

### Example 1.2

Composition of model cerebrospinal fluid: 150 mM Na, 3.0 mM K, 1.4 mM Ca, 0.8 mM Mg, 1.0 mM P, 155 mM Cl (obtained from BiolVT).

### Example 1.3

Composition of peritoneal fluid: DIANEAL (2.27%) CAPD Solution 2.5 L (Baxter).

Bacteria

The following strains of bacteria were inoculated in the model synovial fluid, CSF and peritoneal fluid supplemented with a general purpose medium for growth:
Pseudomonas aeruginosa (Ex 1) (gram negative)
Bacillus cereus (Ex. 2) (gram positive)

Inoculation took place at three levels (CFU/mL): low (1×10¹-1×10³), medium (1×10⁴1×10⁶) and high (1×10⁷-1×10⁹) in 5 mL bottles.

24 hours after inoculation (as a model for a 24 hour post-operative clinical situation) a 250 uL sample of each culture was placed in an Eppendorf tube. Peptide 1 or Peptide A was added to a final concentration of 5 uM. The Eppendorf tube was placed in a DeNiro NIR Fluorimeter (Detact Diagnostics BV, The Netherlands), 24 hours after inoculation as a model for a 24 hour post-operative clinical situation.

The RFU for the control sample (peptide only) was subtracted from each measurement. Above baseline RFU measurement is indicated by (+). No change relative to the baseline is indicated with (−).

TABLE 1
24 hours after inoculation with P. aeruginosa

| | | Peptide 1 | | | Peptide A | | |
|---|---|---|---|---|---|---|---|
| | | | 1 × 10⁴- | 1 × 10⁷- | | 1 × 10⁴- | 1 × 10⁷- |
| | | 1 × 10³ | 1 × 10⁶ | 1 × 10⁹ | 1 × 10³ | 1 × 10⁶ | 1 × 10⁹ |
| 1.1 | Synovial | +++ | +++ | +++ | − | − | − |
| | fluid | | | | | | |
| 1.2 | CSF | ++ | ++ | ++ | − | − | − |
| 1.3 | Peritoneal | +++ | +++ | +++ | − | − | − |
| | fluid | | | | | | |

TABLE 2
24 hours after inoculation with Bacillus cereus

| | | Peptide 1 | | | Peptide A | | |
|---|---|---|---|---|---|---|---|
| | | | 1 × 10⁴- | 1 × 10⁷- | | 1 × 10⁴- | 1 × 10⁷- |
| | | 1 × 10³ | 1 × 10⁶ | 1 × 10⁹ | 1 × 10³ | 1 × 10⁶ | 1 × 10⁹ |
| 2.1 | Synovial | ++ | ++ | ++ | − | − | − |
| | fluid | | | | | | |
| 2.2 | CSF | + | + | + | − | − | − |
| 2.3 | Peritoneal | + | + | + | − | − | − |
| | fluid | | | | | | |

### Example 2

Location of Bacterial Biomarker

The location of the bacterial biomarker in Ex. 1.1 and Ex.2.1 was determined by measuring the fluorescence activity (RFU) of the bacterial cultures, concentrated cells (following centrifugation) and supernatant (following centrifugation).

TABLE 3
RFU activity fractions

| | | Concentrated | |
|---|---|---|---|
| Example | Culture | cells | supernatant |
| P. | 19803 | 878 | 19000 |
| aeruginosa | | | |
| B. cereus | 6363 | 300 | 5838 |

### Example 3

Peptide 2: Peptide cleavage site: ALA; fluorescent agent: IRDye 800CW (Licor Bioscience, USA); non-fluorescent agent: QC-1 (Licor Bioscience, USA)

Peptide B: Peptide cleavage site: AEA; fluorescent agent EDANS; non-fluorescent agent DABCYL.

The detection efficiency of Peptide 2 was compared to a Peptide B.

Example 1.1 and 2.1 were repeated using Peptide 2 and B. Detection took place using a UV-spectrophotometer for Peptides B and C.

TABLE 4
Peptide detection efficiency

| | | P. aeruginosa | B. cereus |
|---|---|---|---|
| | Ratio | inoculation | inoculation |
| Peptide | [b]:([a] + [c]) | 1 × 10⁴ − 1 × 10⁶ | 1 × 10⁴ − 1 × 10⁶ |
| 2 | 1:11 | +++ | ++ |
| B | 1:1.6 | − | − |

### Example 4

Peritoneal dialysis fluid from a patient undergoing automatic peritoneal dialysis overnight, was collected after overnight dialysis. The dwell time of the dialysis fluid is approx. 3 hours. A sample of the waste fluid was collected and spiked with a culture of a clinical isolate of Pseudomonas aeruginosa. The clinical isolate had been collected from a patient with clinical (bacterial) peritonitis.

The level of spiking done at 2 levels: high level consistent with that of a cloudy waste bag (×10⁸ CFU/mL) and low level (×10⁵ CFU/ml) consistent with a clinically relevant amount of bacteria that would lead to infection if not treated.

TABLE 5
Sample Composition

| | |
|---|---|
| Positive sample | Negative sample |

| | | | | |
|---|---|---|---|---|
| Peritoneal fluid volume | 95 | μL | 20 | μL |
| Bacterial culture volume* | 95 | μL | 0 | μL |
| Reagent volume | 10 | μL | 10 | μL |
| Final volume in assay tube | 200 | μL | 200 | μL |

*bacterial culture either at ×10⁸ or ×10⁵ CFU/mL, optionally diluted with sodium phosphate buffer 200 mM, pH 8.75

The reagent (Peptide 3) was synthesised by solid phase peptide synthesis. TQ7WS and TF7WS were purchased from AATBIO (Sunnyvale, CA, USA)amide and PEG1 is Amino-PEG1-acid purchased from BroadPharm (San Diego, CA, USA). Peptide 3 was used at a final concentration in assay tube was 1 uM.
Peptide 3:
N-terminus: TQ7WS
C-terminus: TF7WS
Cleavage site: AFA
Linker: PEG1

Samples were analysed on a TECAN SPARK plate reader using Greiner 96 Flat Black plates and the following settings:
Automatic mirror
Z position=20 000
Gain=175
Shaking 10 s, orbital
Kinetic loop:
Duration: 30 min
Interval type: Fixed 5 min
Fluorescence intensity:
Mode: Top
Excitation wavelength: 756 nm Bandwidth: 5
Emission wavelength: 780 nm Bandwidth: 5

A threshold of 15% relative activity was set for a positive outcome. The threshold was determined as the limit of the blank. The limit of the blank was determined by measuring the fluorescence emission of peptide 3 in phosphate buffer, pH 8.

The ratio of the fluorescence emission intensity (FI) at 780 nm of the positive sample to the negative sample is reported in the table below.

TABLE 6
Sample Composition

| | | Inoculation | Ratio FI positive: |
|---|---|---|---|
| Example | Bacteria | level | negative |
| 4.A | None (negative | none | — |
| | control) | | |
| 4.1 | P. aur | Low | 4 |
| 4.2 | P. aur | High | 10 |
| 4.3 | S. aureus | Low | 3.3 |
| 4.4 | S. aureus | High | 10 |
| 4.5 | P. aur + S. aur | Low | 6.6 |
| 4.6 | P. aur + S. aur | High | 8 |

### Example 5

Briefly, a culture of S. aureus and P. aeruginosa were prepared and used to spike human synovial fluid (Innovate Research, Novi, MI, USA). The samples were spiked to a CFU/mL in the range of. 1×10⁸-1×10¹⁰, which is representative of the bacterial load in infected synovial fluid in periprosthetic joint infection.

Samples were measured 5 minutes after spiking with bacterial culture. Samples were measured using the DeNIRO fluorometer.

The composition of the samples used is shown below in Table 2

TABLE 7
Sample Composition

| | |
|---|---|
| Positive sample | Negative sample |

| | | | | |
|---|---|---|---|---|
| Buffer volume | 130 | μL | 130 | μL |
| Synovial fluid volume | 20 | μL | 20 | μL |
| Bacterial culture volume | 40 | μL | 0 | μL |
| Water volume | 0 | μL | 40 | μL |
| Reagent volume | 10 | μL | 10 | μL |
| Final volume in assay tube | 200 | μL | 200 | μL |

Positive samples gave a ratio of the fluorescence emission intensity (FI) at 780 nm of the positive sample to the negative sample of more than 1.2, whereas the negative samples gave a relative activity below 1 at 5 minutes.

### Example 6

The effect of linkers on the activity of the reagents with an AFA motif was determined in peritoneal dialysate fluid. The composition of the reagents is shown in Table 8. The reagents 6.1-6.5 were screened against a broad-spectrum bacterial protease (P5380, Sigma Aldrich, 4 nM final concentration) as an infection model. A sample of peritoneal dialysate fluid was diluted 50% with sodium phosphate buffer, pH8, mixed with the reagent to a final concentration of peptide of 1 uM in an Eppendorf tube and 200 uL transferred to a microwell plate (Greiner, black, chimney flat bottomed well plate) and the fluorescent emission monitored using a TECAN SPARK plate reader.

Dequenching efficiency was determined by measuring the fluorescent emission intensity at either 780 nm (TF7WS) or 796 nm (IRDye800CW) in the absence (FI¹) and presence of protease (FI²) and determining the percentage change in intensity using the equation: ([(FI²−FI¹)/FI¹]*100

TABLE 8
Results in peritoneal fluid

| | | | | | Dequench- | |
|---|---|---|---|---|---|---|
| | | | | | ing effi- | Ratio |
| | | Link- | Link- | Quench- | ciency | linker:no |
| | Dye | er | er | er | (%) PF¹ | linker |
| 6.1 | TF7WS | None | None | TQ7 | 170 | 1 |
| 6.2 | TF7WS | None | PEG1 | TQ7 | 400 | 2.4 |
| 6.3 | TF7WS | PEG1 | PEG1 | TQ7 | 299 | 1.8 |
| 6.4 | Idye800CW | None | None | QC-1 | 67 | 1 |
| 6.5 | IRDye800CW | None | PEG1 | QC1 | 119 | 1.8 |

¹n = 12

Introduction of one or two PEG1 linkers lead to an unexpected improvement in the clinical performance of the reagent.

### Example 7

The effect of linkers on the activity of the reagents with an AFA motif was determined in synovial fluid. The composition of the reagents is shown in Table 8. The reagents 7.1-7.5 were screened against a broad-spectrum bacterial protease (P5380, Sigma Aldrich 4 nM final concentration) as an infection model in synovial fluid. A sample of synovial fluid was diluted 25% with sodium phosphate buffer, pH8, mixed with the reagent to a final concentration of peptide of 1 uM in an Eppendorf tube and 200 uL transferred to a microwell plate (Greiner, black, chimney flat bottomed well plate) and the fluorescent emission monitored using a TECAN SPARK plate reader The concentration of peptide was 1 uM. The concentration of the protease was 4 nM.

Dequenching efficiency was determined by measuring the fluorescent emission intensity at either 780 nm (TF7WS) or 796 nm (IRDye800CW) in the absence (FI¹) and presence of protease (FI²) and determining the percentage change in intensity using the equation: ([(FI²−FI¹)/FI¹]*100

TABLE 8
Results in synovial fluid

| | | | | | Dequench- | |
|---|---|---|---|---|---|---|
| | | | | | ing effi- | Ratio |
| | | Link- | Link- | Quench- | ciency | linker:no |
| | Dye | er | er | er | (%) SF¹ | linker |
| 7.1 | TF7 | None | None | TQ7 | 103 | 1 |
| 7.2 | TF7 | None | PEG1 | TQ7 | 265 | 2.6 |
| 7.3 | TF7 | PEG1 | PEG1 | TQ7 | 150 | 1.5 |
| 7.4 | Idye800CW | None | None | QC-1 | 35 | 1 |
| 7.5 | IRDye800CW | None | PEG1 | QC1 | 76 | 2.2 |

¹n = 12

Introduction of one or two PEG1 linkers lead to an unexpected improvement in the clinical performance of the reagent.

### Example 8

Peptide 1 was formulated as a freeze-dried lyophilized bead by freeze drying the peptide in the presence of a carbohydrate. Briefly, 25 ug peptide 1 was dissolved in 300 uL PBS, pH 7.4 and added to 300 uL PBS, pH 7.4 containing 20% carbohydrate. 24 beads were dispensed and freeze dried (74 hours, primary temperature −45° C.).

Per excipient, 2 measurements were taken using 2 identical beads. Firstly, in a 0.5 mL microcentrifuge tube, a lyophilized bead was dissolved in 300 uL sterile water at room temperature to prepare the negative sample, the tube was transferred to a DeNIRO® Fluorometer (Detact Diagnostics, Groningen, The Netherlands) and fluorescent emission intensity at 780 nm (TF7WS) recorded. Secondly, in a 0.5 mL microcentrifuge tube, a lyophilized bead was dissolved in 300 uL a solution of subtilisin (4 nM) in sterile water to prepare the positive sample. The mixture was briefly vortexed, incubated at 21° C. for 15 minutes and then fluorescent emission intensity at 780 nm (TF7WS) measured using the DeNIRO® Fluorometer.

The ratio of the fluorescence emission intensity (FI) at 780 nm of the positive sample to the negative sample is reported in the table below.

TABLE 9
Results

| | | FI | FI | Ratio |
|---|---|---|---|---|
| | Carbohydrate | Negative | Positive | RFU2:RFU1 |
| 7 A | None | 12101 | 16451 | 1.4 |
| 7.1 | Dextran¹ | 6794 | 27044 | 4 |
| 7.2 | Trehalose² | 13293 | 23717 | 2 |
| 7.3 | Lactose³ | 11725 | 24244 | 2.1 |
| 7.4 | Mannitol⁴ | 11528 | 16888 | 1.5 |

¹Dextran: Sigma cat 31388;
²Trehalose: Life Sciences cat TDH033;
³Lactose: Sigma cat L3750;
⁴Mannitol: Sigma cat M4125

Table 9 shows that all formulations containing carbohydrate gave an unexpected improvement in relative activity. Surprisingly, lyophilized beads comprising a polysaccharide, dextran, showed an unexpected stabilization of the background (lowest negative FI) and the highest ratio of positive to negative samples at 780 nm.

### Example 10

Lyophilized beads comprising dextran according to Example 9 were dissolved 200 uL of bodily fluid (peritoneal dialysis fluid and synovial fluid as shown in Table 11). The bacterial cultures were prepared as per Example 4.

TABLE 10
Sample composition

| | |
|---|---|
| Positive sample | Negative sample |

| | | | | |
|---|---|---|---|---|
| Buffer volume | 140 | μL | 140 | μL |
| Bodily fluid volume | 20 | μL | 20 | μL |
| Bacterial culture volume | 40 | μL | 0 | μL |
| Water volume | 0 | μL | 40 | μL |
| Final volume in assay tube | 200 | μL | 200 | μL |

TABLE 11
Results

| | | Positive | Negative |
|---|---|---|---|
| | Clinical Sample | sample | sample |
| 8.1 | Peritoneal dialysis | >threshold | <threshold |
| | fluid | | |
| 8.2 | Synovial fluid | >threshold | <threshold |

### Comparative Example

Clinical samples of peritoneal fluid and synovial fluid were spiked with a culture of S. aureus as per Example 4 and 5 and subjected to an acidification step as described by Example 1 of WO2018/224561 prior to determining the dequenching efficiency. 1 mL of clinical sample was adjusted to pH 4.6 by addition of 10% acetic acid. Solutions were kept at room temperature for 15 minutes. 0.1 mL 1 M sodium acetate buffer was added and the samples were centrifuged for 30 minutes at 4500 g. When the samples were analysed as per Example 10, no measurable activity above the threshold was found.

1-25. (canceled)

26. A lyophilized bead comprising a reagent and a carbohydrate, the reagent having the general formula (a)−(b)−(c) (I), wherein:
(a) is a fluorescent agent having an emission wavelength of 650-900 nm,
(c) is a non-fluorescent agent having an absorption wavelength of 650-900 nm, for quenching said emission of said fluorescent agent, and
(b) is a peptide comprising a cleavage site, the cleavage site being specific for a bacterial biomarker, wherein the bacterial biomarker is a bacterial membrane bound protease, bacterial membrane bound transpeptidase, intracellular bacterial protease or extracellular bacterial protease, and
the carbohydrate being chosen from the group consisting of monosaccharide, disaccharide, polysaccharide and combinations thereof.

27. The lyophilized bead according to claim 26, wherein the mass ratio of (b):((a)+(c)) is at least 1:2.

28. The lyophilized bead to claim 26, wherein the reagent has the general formula (a)−(linker1)ₙ−(b)−(linker2)ₘ−(c), wherein n is 0, 1 or 2, m is 0, 1 or 2, and (linker1) and (linker2) are independently selected from the group of an optionally substituted hydrocarbyl group and a non-proteolytic hydrocarbyl group.

29. The method according to claim 26, wherein the fluorescent agent having an emission wavelength of 650-900 nm comprises a cyanine moiety, and wherein the non-fluorescent agent having an absorption wavelength of 650-900 nm comprises a cyanine moiety.

30. The lyophilized bead according to claim 26, wherein the bacterial biomarker is a bacterial membrane bound transpeptidase or an extracellular bacterial protease.

31. The lyophilized bead according to claim 26, wherein the cleavage site is specific for a bacterial biomarker for a bacteria species selected from the group consisting of Bacillus cereus, Staphylococcus aureus, Streptococcus pyogenes, Pseudomonas aeruginosa, Escherichia coli and combinations thereof.

32. The lyophilized bead according to claim 26, wherein (b) comprises between 3 and 8 amino acids.

33. The lyophilized bead according to claim 26, wherein (b) has the general formula Xₐₐ₁-Xₐₐ₂-Xₐₐ₃, wherein:
Xₐₐ₁ is a small, hydrophobic amino acid;
Xₐₐ₂ is a small or aromatic hydrophobic amino acid;
Xₐₐ₃ is selected form the group consisting of alanine, glycine, leucine, valine, norleucine, norvaline, isoleucine, isovaline, alloisoleucine.

34. The lyophilized bead according to claim 26, wherein the bead comprises 5 to 95 wt. % by weight of the bead carbohydrate.

35. The lyophilized bead of claim 26, wherein the carbohydrate is selected from the group consisting of
the monosaccharides glucose, fructose, galactose and combinations thereof;
the disaccharides sucrose, maltose, lactose, trehalose and combinations thereof; and
the polysaccharides alginate, chitosan, hyaluronic acid, cellulose derivatives, dextran and combinations thereof.

36. A method for in vitro diagnosing infection in bodily fluid samples using beads according to claim 26, wherein the method comprises the steps of:
i) contacting a sample of bodily fluid, preferably a human bodily fluid, with the beads comprising the reagent in a sample receptacle, and
ii) monitoring the fluorescence emission in the range of 650-900 nm from the sample in the receptacle in step i), wherein an increase in fluorescence emission in the range of 650-900 nm is indicative for the presence of infection in the bodily fluid sample.

37. The method according to claim 36, wherein step i) comprises the step of adding a diluent.

38. The method according to claim 37, wherein the volume ratio of diluent to sample is in the range of 0.1 to 100.

39. The method according to claim 36, wherein the concentration of reagent in the sample receptacle after addition of the sample, and optionally a diluent, is in the range of 0.01 to 10 μM.

40. The method according to claim 36, wherein the sample is not subjected to an ex vivo step of bacterial incubation in the temperature range of 20 to 40° C. either prior to or after being contacted with the reagent.

41. The method according to claim 36, wherein the method comprises the step of contacting the sample with the beads comprising the reagent and second beads having a second reagent and said carbohydrate, the second reagent having the general formula (a)−(d)−(c) (III), wherein (d) is a peptide comprising a cleavage site that is different from the cleavage site of the peptide (b).

42. A kit for in vitro diagnosing infection in bodily fluid samples, comprising:
a) a container comprising a lyophilized bead of claim 26,
b) a set of instructions, and
c) optionally a container containing a diluent.

43. The kit according to claim 42, wherein the reagent and the diluent are present in two compartments of a container.

44. A system comprising:
a) a container comprising a lyophilized bead of claim 26, and
b) a device adapted to receive the container and monitor the fluorescence signal emitted from the reagent in the presence of a sample of synovial fluid, peritoneal fluid or cerebrospinal fluid with the reagent.

45. The system according to claim 44, wherein the system is a point-of-care system or a patient beside system.
